(19) United States
(12) Patent Application Publication
(10) Pub. No.: US 2002/0062494 A1
(43) Pub. Date: May 23, 2002

(54) Method for increasing transgenic biomass

(76) Inventor: Phillippe Bournat, Clermond Ferrand (FR)

Correspondence Address: PALMER & DODGE, LLP, KATHLEEN M. WILLIAMS / STR, 111 HUNTINGTON AVENUE, BOSTON, MA, 02199, US

(21) Appl. No.: 09/973,122

(22) Filed: Oct. 9, 2001

(30) Foreign Application Priority Data
Oct. 12, 1999 (FR) 99/12797

(51) Int. Cl. A01H 1/02; A01H 5/00; A01H 1/00; C12N 15/82

(52) U.S. Cl. 800/271; 800/260; 800/278; 800/274; 800/320.1

(57) ABSTRACT

The present invention relates to a method for rapidly increasing the amount and the quality of a transgenic biomass, in particular in the production of transgenic plants which produce a recombinant protein for therapeutic use, and applies more particularly to maize.

### FIELD OF THE INVENTION

The present invention relates to a method for increasing the amount and the quality of a plant transgenic biomass.

### BACKGROUND OF THE INVENTION

The expression of recombinant proteins for therapeutic use in plants is often delayed due to the time required for obtaining the plant raw material containing the recombinant protein.

This problem is all the more significant in certain plant species such as maize, given that, on the one hand, the time required for obtaining the primary transformants is relatively long and, on the other hand, the organs in which the expression of the recombinant protein occurs, in this case the seeds, are produced in a small amount. The use of this type of plant thus requires multiplication phases prior to the purification and extraction of the recombinant protein produced. These multiplication phases are detrimental to project dynamics.

The object of this invention is to increase the biomass obtained in the first generation, and thus to decrease the number of multiplication phases required for obtaining a large biomass. This would make it possible to very significantly shorten research and development programme delays, and to obtain, relatively inexpensively, a large amount of plant raw material or biomass, also required for the analysis of the conformity of the recombinant protein of interest.

Seed multiplication is a logarithmic phenomenon: at each generation there is about a one hundred-fold multiplication of the number of seeds obtained for the classical transformable genotypes, the agronomic value of which is low. Depending on the crossing techniques used, backcross or self-pollination, a variable proportion of the seeds formed will contain the gene of interest.

The applicant has found that the solution to this problem lies, in part, in increasing the initial pool of seeds before sexual multiplication, which makes it possible to gain a multiplication cycle and to very rapidly obtain a satisfactory amount of biomass for experiments and recombinant protein production in the plants of the type previously described.

### SUMMARY OF THE INVENTION

One object of the present invention is a method for increasing transgenic plant biomass comprising the steps of:

(a) providing plant calluses which contain at least one genetic transformation event and which are capable of regeneration;

(b) multiplying the plant calluses;

(c) regenerating whole transgenic T0 plants, from the plant calluses;

(d) pollinating the whole transgenic T0 plants with non-transgenic pollen to produce T1 seeds;

(e) harvesting the T1 seeds which have integrated at least one transgene of interest;

(f) sowing the transgenic T1 seeds and pollinating the plants which result therefrom, wherein pollination is by self-pollination or by free pollination, to produce T2 seeds; and

(g) harvesting the T2 transgenic seeds.

In a preferred embodiment, the method further comprises the step of selecting the calluses which comprise at least one genetic transformation event of interest, prior to the regeneration step (c).

As used herein, “transgenic” refers to a plant, plant cell, or multitude of structured or unstructured plant cells having integrated, via well known techniques of genetic manipulation and gene insertion, a sequence of nucleic acid representing a gene of interest into the plant genome, and typically into a chromosome of a cell nucleus, mitochondria or other organelle containing chromosomes, at a locus different to, or in a number of copies greater than, that normally present in the native plant or plant cell. Transgenic plants result from the manipulation and insertion of such nucleic acid sequences, as opposed to naturally occurring mutations, to produce a non-naturally occurring plant or a plant with a non-naturally occurring genotype. Techniques for transformation of plants and plant cells are well known in the art and may comprise for example electroporation, microinjection, agrobacterium mediated transformation, and ballistic transformation.

As used herein, “biomass” refers to useful biological material including a product of interest, which material is to be collected and is intended for further processing to isolate or concentrate the product of interest. “Biomass” is typically comprised of the fruit of a plant, for example its seeds, but may also comprise the leaves where these are the parts of the plant that contain a product of interest. “Biomass”, as it refers to plant material, includes any structure or structures of a plant that contain a product of interest or can express a product of interest.

As used herein, a “product of interest” includes a molecule that is to be produced by a transgenic plant of the invention. Preferably, a “product of interest” is a “protein of interest” produced from a “gene of interest”.

As used herein, “increasing” refers to producing more. For example, “increasing the biomass” refers to producing more biomass as compared to the amount of biomass produced by a conventional method that does not include a step wherein the initial pool of seeds is increased prior to sexual multiplication. An “increase” refers to at least 1-fold, preferably 1-5 fold and more preferably 5-fold or more, (for example 6-fold, 10-fold, 100-fold, 1000-fold) as compared to the amount of biomass produced by conventional techniques, well-known in the art.

As used herein, “plant calluses” are groups of structured or unstructured undifferentiated plant cells. An “unstructured undifferentiated” plant callus is one in which no plant structures are discernible in the callus by one of skill in the art, at a magnification of no greater than 120X. In contrast, a “structured” plant callus is one in which specific plant structures, including, but not limited to, axial structures, such as a root, shoot, stem, or leaf structures, are discernible by one of skill in the art when observed at a magnification of no greater than 120X. The minimum number of cells present in a “plant callus is the number of cells required such that one of skill in the art would be capable of separating the “plant callus” from the surrounding cells under a maximum magnification of 120X.

As used herein, “multiplying plant calluses” refers to growing the plant calluses on a suitable growth media known to those skilled in the art, e.g. Murashige Skoog media, to enable the cells to divide and multiply, and, where appropriate, differentiate.

As used herein, “multiply” refers to increase in number by at least two fold and preferably 2-fold or more (for example, 3, 4, 5,10,100, 1000-fold etc . . . ).

As used herein, “differentiation refers to the process by which a cell undergoes a change to a particular cell type, e.g. to a specialized cell type.

As used herein, a “genetic transformation event of interest” refers to an integration event of one or more nucleic acid sequences of interest that lead to the expression of the gene or genes coded by the integrated sequence or sequences and which it is desired to produce. The invention provides for the integration of a nucleic acid sequence of a gene of interest and/or a nucleic acid sequence that encodes for a protein of interest.

As used herein, “protein of interest” refers to any protein that is either heterologous or endogenous to the plant used in the method of the invention.

As used herein, “gene of interest” refers to any gene that is either heterologous or endogenous to the plant used in the method of the invention.

As used herein, “nucleotide sequence of interest” refers to any nucleotide sequence that is either heterologous or endogenous to the plant used in the method of the invention.

“Heterologous” refers to a gene which is not naturally present in the genome of the plant used in the method of the invention or a gene which is not naturally present in a plant genome. “Heterologous” also refers to a protein which is not naturally expressed from the genome of the plant used in the method of the invention or a plant genome. p “Endogenous” refers to a gene which is naturally present in the genome of the plant used in the method of the invention or a plant genome. “Endogenous” also refers to a protein which is naturally expressed from the genome of the plant used in the method of the invention or a plant genome.

As used herein, “regeneration” refers to the process of producing a new growth of cells and tissues. In one embodiment, “regeneration” according to the invention results in the formation of a whole plant.

As used herein, “capable of regeneration” refers to transgenic plant parts, transgenic plant cells or transgenic plant calluses that are capable of regeneration into whole plants when grown on suitable growth media known to the skilled person.

As used herein, “selecting the calluses” refers to choosing a callus for regeneration into a whole plant. Many selection methods are known per se to the skilled person, including selection through genetic integration of a gene coding for antibiotic or herbicide resistance. Such selection is frequently carried out by growing the calluses on a growth media containing an antibiotic, e.g. kanamycin, neomycin, or hygromycin, or a herbicide, e.g. glyphosate or glufosinate, that will inhibit growth in those plants that do not bear and express the gene coding for the antibiotic or herbicide resistance, since those that have integrated the gene for resistance will be tolerant to such media and will survive.

As used herein, “regenerating whole transgenic plants” refers to the regeneration of a whole plant from a plant callus, wherein regeneration comprises the steps of differentiating a cell into roots, shoots and leaves to form a plantlet, and then growing the plantlet, initially in vitro, and then in soil following transfer of the plantlet at a predetermined stage of maturity, e.g. 6 to 15 weeks after transformation.

As used herein, “whole transgenic plants”, ‘refers to the viable plants obtained through regeneration of transgenic plant parts, transgenic plant cells or transgenic plant calluses as described previously.

As used herein, “pollinating” refers to the fertilization of a female part of a plant with pollen.

As used herein, “self-pollination” is the process by which fertilization occurs when pollen from a male part of a plant comes into contact with the female part of the same individual plant.

As used herein, “free pollination” is the process by which fertilization occurs when pollen from the male part of one plant comes into contact with the female part of a different plant.

As used herein, “non-transgenic pollen” refers to pollen that does not bear a genetic transformation event of interest, or does not originate from a transgenic plant.

As used herein, “harvesting” refers to the collection of a biomass, by manual or mechanical means; such as with a combine harvester.

As used herein, “seeds” refers to the fruit of a plant, generally bearing the genetic information and including the biochemical resources necessary for usual regeneration of a whole plant therefrom.

As used herein, “T1 seeds” refers to the seeds obtained at the end of the first cycle of transformation and regeneration of plants from transgenic calluses.

As used herein, “T2 seeds” refers to the seeds obtained at the end of the second cycle of plant regeneration.

As used herein, “integrated” refers to the stable inclusion of a nucleic acid sequence representing a gene into a locus on a plant chromosome.

As used herein, “sowing” refers to the insertion of a seed into the soil or other suitable growth media, such as growbags, or in vitro nutrient media.

As used herein, “primary transformants” and “T0 plants” are the plants obtained at the end of the regeneration step from transgenic calluses. Such plants yield T1 seeds as defined above.

In a preferred embodiment, the method further comprises the step of post-harvest phenotypic sorting of the T2 seeds, prior to the regeneration step.

As used herein, “post harvest phenotypic sorting” refers to the sorting of the biomass according to a visible, phenotypic, characteristic that distinguishes one plant from the next, such as grain size, shape or color.

In another preferred embodiment, the post-harvest phenotypic sorting is carried out on T2 seeds originating from a plant used only as a female.

As used herein, a “plant used only as a female” refers to a plant that is used only to produce seed after fertilization, in which only the female part of the plant is operational.

As used herein, a “plant used only as a male” refers to a plant that is used only for the production of pollen, in which only the male part of the plant is operational.

In another preferred embodiment, the transgenic T2 seeds have a coloured phenotype which is different from the non-transgenic seeds.

In another preferred embodiment, the method of increasing transgenic biomass utilizes plants used only as male plants and plants used only as female plants.

In another preferred embodiment, the T2 seeds originating from the plants used as male plants are harvested independently from the T2 seeds originating from the plants used as female plants.

In another preferred embodiment, the plant is allogamous.

As used herein, “allogamous” refers to a plant that is mainly fertilized by another plant.

In another preferred embodiment, the plant is maize.

As used herein, “mainly fertilized” refers to fertilization in which at least 50% (for example, 50%, 51%, 55%, 60%, 75%, or 100%) of the fertilization events are via another plant. Preferably, at least 70%, 80% and up to 90% of the fertilization events are via another plant.

In another preferred embodiment, the primary transformants are pollarded or castrated before pollination with non-transgenic pollen.

As used herein, “pollard” or “castrate” refers to cutting off the parts of a plant bearing the male part or pollen.

In another preferred embodiment, the multiplication step results in the production of at least 10, and preferably at least 20 transgenic plants comprising each genetic transformation event.

For example, in the case of maize, and in order to obtain maize calluses comprising one or more transgenes, it is possible to use the technique of transforming the maize using an agrobacterium, by involving immature embryos. This technique involves a regeneration phase which gives rise to transformants which can be copied effectively. This copying, which is relatively easy to carry out, makes it possible to obtain, in vitro, young transformed T0 plants which are strictly identical with respect to the transgene. These plants, which are isolated visually, are, with good reliability, copies of the initial plant. The extra work involved corresponds to cloning work, to culturing all the clones in a phytotron and then in a greenhouse, and to controlling, for example, by molecular analysis, the identity of the valuable clones. This cloning can be carried out on all the primary transformants, and then, after a biochemical screen, for example, only the most valuable (strongest expression in accordance with cleanness of the inserts) will be maintained.

In another preferred embodiment, the transgenic T1seeds are sown, cultivated and used as male plants.

In another preferred embodiment, the transgenic T1 seeds are sown in a line alternating, preferably in a 4/2 or in a 6/2 configuration, with non-transgenic plants used as female plants. Preferably, the T1 seeds to be used as male plants are sown in a line alternating with non-transgenic seeds to be used as female plants.

As used herein, “sown in a line” refers to the sowing of seeds in a line, such that the plants that grow therefrom form an aligned row.

As used herein, “4/2” refers to a sowing method in which two rows of plants used only as male and four rows of plants used only as female are sown alternately, and “6/2” refers to a sowing method in which two rows of plants used only as male and six rows of plants used only as female are sown alternately.

In another preferred embodiment, the female transgenic plants are sterile male plants.

As used herein, “sterile male plants” refers to plants that are unable to produce pollen.

In another preferred embodiment, the female non-transgenic plants are castrated.

In another preferred embodiment, the female plants have a high agronomic value as compared to the male plants.

As used herein, “high agronomic value” refers to a type or variety of plant that represents a significant interest for a farmer in terms of seed quality, crop yield, plant vigorousness and economic and physiological suitability of the plant to grow in the climatic conditions of its geographical area of cultivation.

### DETAILED DESCRIPTION OF THE INVENTION

The invention provides for a method for increasing the transgenic plant biomass. The invention provides for a method of amplifying the regeneration number obtained from a genetic transformation event. The invention also provides for a method of providing a pollen mass which is of a size sufficient to pollinate non-transgenic plants with a minimum amount of work. The invention also provides for a method of rapidly identifying a biomass comprising a transgene of interest wherein the transgene of interest is introduced along with a gene which confers a phenotypic nature which enables post-harvest industrial sorting.

Plants:

Plants that can be used in accordance with the present invention can be any monocotyledonous or dicotyledonous plant. Preferably a plant of the invention is a monocotyledon. The types of plant that can be used are generally well known to the skilled person, and include, but are not limited to tobacco, potato, lettuce, sunflower, beetroot, rape, canola, wheat, barley, rice, oats and corn, with corn being the most preferred.

Plants that can be used for practice of the present invention also include, but are not limited to, carrot, spinach, pepper, tomato, apple, rye, soybean, maize, berries such as strawberries, raspberries, alfalfa and banana.

Vectors Useful According to the Invention

The invention provides for nucleic acid (DNA or RNA) constructs comprising a nucleotide sequence encoding a protein of interest. In certain embodiments, a construct of the invention additionally comprises a marker gene encoding for antibiotic or herbicide resistance.

A DNA construct according to the invention preferably has a plant-functional promoter operably linked to the 5′ end of a nucleotide sequence of interest. A preferred promoter is selected from among CaMV 35S, tomato E8, patatin, ubiquitin, mannopine synthase (mas), rice actin 1, soybean seed protein glycinin (Gyl), soybean vegetative storage protein (vsp), and granule-bound starch synthase (gbss). A construct of the present invention can also include a translational enhancer region, such as tobacco etch virus (TEV) enhancer, which has been described elsewhere (Carrington, et al. (1990)). Optionally, a construct of the invention can comprise at least one vegetative storage protein (VSP) signal peptide encoding sequence, such as an αS or αL sequence (Mason et al 1988), operably linked to the 5′ end of a nucleotide sequence encoding a protein of interest.

As used herein, the term “operably linked” refers to the respective coding sequence being fused in-frame to a promoter, enhancer, termination sequence, and the like, so that the coding sequence is faithfully transcribed, spliced, and translated, and the other structural features are able to perform their respective functions.

A nucleotide sequence of interest of the invention is preferably operably linked at its 3′ end to a plant-functional termination sequence. Preferred termination sequences include nopaline synthase (nos), vegetative storage protein (vsp), protease inhibitor 2 (pin2), and geminiviral short intergenic (sir) termination sequences.

A DNA construct of the invention can be single-stranded or in its double-stranded replicative form, which includes a complementary strand. As used herein, the term “transgene” refers to a nucleotide sequence encoding a protein of interest together with the regulatory features necessary to effect transcription of the coding sequence. Such a transgene can be synthesized directly or derived from a genomic or cDNA library, and additionally may be amplified, such as by the polymerase chain reaction (PCR), according to methods well known in the art and described in Maniatis, supra, Ausubel, supra).

Another aspect of the present invention is an expression vector comprising an aforementioned DNA construct of the invention. Such a vector includes a selectable marker gene and a multiple cloning site into which is inserted a nucleotide sequence encoding a protein of interest. An expression vector of the invention also preferably has an E. coli origin of replication, in order to permit the use of conventional techniques in producing clones of the construct.

Marker genes useful according to the invention may include a gene encoding a selectable marker, e.g., an antibiotic resistance gene such as the bacterial tetracycline resistance gene. Incorporation of the tetracycline resistance gene permits the use of tetracycline as a selective agent in the plasmid preparation procedure according to the invention. One advantage to the use of a tetracycline resistance gene is that tetracycline is not degraded in E. coli, and therefore more tetracycline does not have to be added during fermentation. In addition, the tetracycline resistance gene is preferred over a gene encoding ampicillin resistance because tetracycline is prescribed less often as an antibiotic in a clinical setting, and therefore read through from the plasmid resistance gene will be less likely to interfere with the use of an antibiotic in a clinical setting.

Additional marker genes useful according to the invention include resistance to biocide, particularly an antibiotic, such as kanamycin, G418, bleomycin, hygromycin, chloramphenicol or the like. The particular marker employed will be one which allows for selection of transformed cells as compared to cells lacking the nucleic acid which has been introduced. Additional marker genes useful according to the invention also include herbicide resistance genes.

A vector can also have an A. tumefaciens origin of replication, such as when it is desired to maintain the vector in A. tumefaciens for later transformation with this system. In this event, the nucleotide sequence encoding a protein of interest is flanked by the left and right T-DNA border regions to effect its transfer to a host plant cell.

As used herein, the term “vector”, and the like, refers to a nucleic acid construct capable of self-replication. Such a vector includes a plasmid, bacteria transformed with plasmids, phage vectors, cosmids, and bacterial and yeast artificial chromosomes. Generally, a vector of the present invention will be a plasmid, whether it is present in vitro, in E. coli, in A. tumefaciens , or as a nuclear episome of a plant. Suitable techniques for assembling the instant structural components into an expression cassette or replicon are described by Maniatis et al. (1982).

A strain of bacteria, such as E. coli, can be transfected with an expression vector of the present invention in order to grow/amplify an instant expression cassette according to methods well known in the art (Ausubel, supra, Maniatis, supra). The E. coli can also be mated with A. tumefaciens to introduce the vector therein, where it can reside intact as a shuttle vector. A helper Ti plasmid in the A. tumefaciens can provide the vir genes necessary to transfer the T-DNA directly from the shuttle vector to the plant cell. Alternatively, the vector can undergo homologous recombination with a tumor-inducing (Ti) plasmid and exchange the instant cassette for the T-DNA of the Ti plasmid. The invention therefore provides for producing transiently transformed plant cells wherein DNA constructs are maintained as episomes. Alternatively, the invention provides for methods of stably transforming plant cells wherein a DNA construct that is introduced into a plant cell is stably integrated into a chromosome.

Methods of Gene Transfer into Plants

Introduction of DNA into protoplasts of a plant can be effected by treatment of the protoplasts with an electric pulse in the presence of the appropriate DNA in a process called electroporation. In this method, the protoplasts are isolated and suspended in a mannitol solution. Supercoiled or circular plasmid DNA is added. The solution is mixed and subjected to a pulse of about 400 V/cm at room temperature for less than 10 to 100 microseconds. A reversible physical breakdown of the membrane occurs to permit DNA uptake into the protoplasts.

DNA viruses have been used as gene vectors in plants. A cauliflower mosaic virus carrying a modified bacterial methotrexate-resistance gene was used to infect a plant. The foreign gene was systematically spread in the plant [Brisson, N. et al., Nature 310, 511 (1984)]. The advantages of this system are the ease of infection, systematic spread within the plant, and multiple copies of the gene per cell.

Liposome fusion has also been shown to be a method for transformation of plant cells. In this method, protoplasts are brought together with liposomes carrying the desired gene. As membranes merge, the foreign gene is transferred to the protoplasts [Dehayes, A. et al, EMBO J. 4, 2731 (1985)].

Polyethylene glycol (PEG) mediated transformation has been carried out in N. tabacum (a dicot) and Lolium multiflorum (a monocot). It is a chemical procedure of direct gene transfer based on a synergistic interaction between Mg²⁺, PEG, and possibly Ca²⁺[Negrutiu, R. et al., Plant Mol. Biol. 8, 363 (1987)]. Alternatively, exogenous DNA can be introduced into cells or protoplasts by microinjection. A solution of plasmid DNA is injected directly into the cell with a finely pulled glass needle.

A recently developed procedure for direct gene transfer involves bombardment of cells by microprojectiles carrying DNA [Klein, T. M. et al, Nature 327, 70 (1987)]. In this “biolistic” procedure, tungsten or gold particles coated with the exogenous DNA are accelerated toward the target cells. At least transient expression has been achieved in onion. This procedure has been utilized to introduce DNA into Black Mexican sweet corn cells in suspension culture and maize immature embryos and also into soybean protoplasts [Klein, T. M. et al., Bio/Technology 6, 559 (1988)]. Stably transformed cultures of maize and tobacco have been obtained by microprojectile bombardment. Stably transformed soybean plants have been obtained by this procedure [McCabe, D. E. et al., Bio/Technology 6, 923 (1988)].

To produce transformed seeds, flowers of Arabidopsis are transformed according to the following method. The Agrobacterium is vacuum-infiltrated into developing flowers, and the resulting seed are then screened for marker resistance and foreign gene expression. Presumably, stamens/pollen, ovary/egg, or even the developing zygote if fertilization has already occurred are transformed. This method (described in Clough & Bent, 1998, Plant J., 16:735) is used to transform Arabidopsis with a construct comprising a rep gene under the transcriptional regulation of the At2S-2 seed promoter.

Methods of Detecting Nucleic Acid and Protein According to the Invention

The invention provides for methods of detecting a nucleic acid of interest including but not limited to Southern and northern blot analysis, PCR-based methods of detection, as well as immunological methods of detecting a protein of interest according to the invention.

A. Detection of a Nucleotide Sequence of Interest

1. Southern Blot Analysis

Southern blot analysis can be used to detect a nucleotide sequence of interest from a PCR amplified product or from a total genomic DNA test sample via a non-PCR based assay. The method of Southern blot analysis is well known in the art (Ausubel et al., supra, Sambrook et al., 1989, Molecular Cloning. A Laboratory Manual., 2nd Edition, Cold Spring Harbor Laboratory Press, Cold Spring Harbor, N.Y.). This technique involves the transfer of DNA fragments from an electrophoresis gel to a membrane support resulting in the immobilization of the DNA fragments. The resulting membrane carries a semipermanent reproduction of the banding pattern of the gel.

Southern blot analysis is performed according to the following method. Genomic DNA (5-20 μg) is digested with the appropriate restriction enzyme and separated on a 0.6-1.0% agarose gel in TAE buffer. The DNA is transferred to a commercially available nylon or nitrocellulose membrane (e.g. Hybond-N membrane, Amersham, Arlington Heights, Ill.) by methods well known in the art (Ausubel et al., supra, Sambrook et al., supra). Following transfer and UV cross linking, the membrane is hybridized with a radiolabeled probe in hybridization solution (e.g. under stringent conditions in 5X SSC, 5X Denhardt solution, 1% SDS) at 65 CAlternatively, high stringency hybridization can be performed at 68 Corin a hybridization buffer containing a decreased concentration of salt, for example 0.1X SSC. The hybridization conditions can be varied as necessary according to the parameters known in the art. Following hybridization, the membrane is washed at room temperature in 2X SSC/0.1% SDS and at 65 Gh 0.2X SSC/0.1% SDS, and exposed to film. The stringency of the wash buffers can also be varied depending on the amount of the background signal (Ausubel et al., supra).

Detection of a nucleic acid probe-target nucleic acid hybrid will include the step of hybridizing a nucleic acid probe to the DNA target. This probe may be radioactively labeled or covalently linked to an enzyme such that the covalent linkage does not interfere with the specificity of the hybridization. A resulting hybrid can be detected with a labeled probe. Methods for radioactively labeling a probe include random oligonucleotide primed synthesis, nick translation or kinase reactions (see Ausubel et al., supra). Alternatively, a hybrid can be detected via non-isotopic methods. Non-isotopically labeled probes can be produced by the addition of biotin or digoxigenin, fluorescent groups, chemiluminescent groups (e.g. dioxetanes, particularly triggered dioxetanes), enzymes or antibodies. Typically, non-isotopic probes are detected by fluorescence or enzymatic methods. Detection of a radiolabeled probe-target nucleic acid complex can be accomplished by separating the complex from free probe and measuring the level of complex by autoradiography or scintillation counting. If the probe is covalently linked to an enzyme, the enzyme-probe-conjugate-target nucleic acid complex will be isolated away from the free probe enzyme conjugate and a substrate will be added for enzyme detection. Enzymatic activity will be observed as a change in color development or luminescent output resulting in a 10³-10⁶ increase in sensitivity. An example of the preparation and use of nucleic acid probe-enzyme conjugates as hybridization probes (wherein the enzyme is alkaline phosphatase) is described in (Jablonski et al., 1986, Nuc. Acids Res., 14:6115).

Two-step label amplification methodologies are known in the art. These assays are based on the principle that a small ligand (such as digoxigenin, biotin, or the like) is attached to a nucleic acid probe capable of specifically binding to a gene of interest.

According to the method of two-step label amplification, the small ligand attached to the nucleic acid probe will be specifically recognized by an antibody-enzyme conjugate. For example, digoxigenin will be attached to the nucleic acid probe and hybridization will be detected by an antibody-alkaline phosphatase conjugate wherein the alkaline phosphatase reacts with a chemiluminescent substrate. For methods of preparing nucleic acid probe-small ligand conjugates, see (Martin et al., 1990, BioTechniques, 9:762). Alternatively, the small ligand will be recognized by a second ligand-enzyme conjugate that is capable of specifically complexing to the first ligand. A well known example of this manner of small ligand interaction is the biotin avidin interaction. Methods for labeling nucleic acid probes and their use in biotin-avidin based assays are described in Rigby et al., 1977, J. Mol. Biol., 113:237 and Nguyen et al., 1992, BioTechniques, 13:116).

Variations of the basic hybrid detection protocol are known in the art, and include modifications that facilitate separation of the hybrids to be detected from extraneous materials and/or that employ the signal from the labeled moiety. A number of these modifications are reviewed in, e.g., Matthews & Kricka, 1988, Anal. Biochem., 169:1; Landegren et al., 1988, Science, 242:229; Mittlin, 1989, Clinical Chem. 35:1819; U.S. Pat. No. 4,868,105, and in EPO Publication No. 225,807.

2. Northern Blot Analysis

The method of Northern blotting is well known in the art. This technique involves the transfer of RNA from an electrophoresis gel to a membrane support to allow the detection of specific sequences in RNA preparations.

Northern blot analysis is performed according to the following method. An RNA sample (prepared by the addition of MOPS buffer, formaldehyde and formamide) is separated on an agarose/formaldehyde gel in 1X MOPS buffer. Following staining with ethidium bromide and visualization under ultra violet light to determine the integrity of the RNA, the RNA is hydrolyzed by treatment with 0.05M NaOH/1.5MNaCl followed by incubation with 0.5M Tris-Cl (pH 7.4)1.5M NaCl. The RNA is transferred to a commercially available nylon or nitrocellulose membrane (e.g. Hybond-N membrane, Amersham, Arlington Heights, Ill.) by methods well known in the art (Ausubel et al., supra, Sambrook et al., supra). Following transfer and UV cross linking, the membrane is hybridized with a radiolabeled probe in hybridization solution (e.g. in 50% formamide/2.5% Denhardt's/100-200 mg denatured salmon sperm DNA/0.1% SDS/5X SSPE) at 42 CThe hybridization conditions can be varied as necessary as described in Ausubel et al., supra and Sambrook et al., supra. Following hybridization, the membrane is washed at room temperature in 2X SSC/0.1% SDS, at 42 C in 1X SSC/0.1% SDS, at 65 Cn0.2X SSC/0.1% SDS, and exposed to film. The stringency of the wash buffers can also be varied depending on the amount of background signal (Ausubel et al., supra).

3. PCR

Nucleic acid sequences of interest of the invention are amplified from genomic DNA or other natural sources by the polymerase chain reaction (PCR). PCR methods are well-known to those skilled in the art.

PCR provides a method for rapidly amplifying a particular DNA sequence by using multiple cycles of DNA replication catalyzed by a thermostable, DNA-dependent DNA polymerase to amplify the target sequence of interest. PCR requires the presence of a nucleic acid to be amplified, two single stranded oligonucleotide primers flanking the sequence to be amplified, a DNA polymerase, deoxyribonucleoside triphosphates, a buffer and salts.

The method of PCR is well known in the art. PCR, is performed as described in Mullis and Faloona, 1987, Methods Enzymol., 155: 335, herein incorporated by reference.

PCR is performed using template DNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of 10 □ PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30 C and 72 C is used. Initial denaturation of the template molecules normally occurs at between 92 C and 99 Cfor 4 minutes, followed by 20-40 cycles consisting of denaturation (94-99 C for 15 seconds to 1 minute), annealing (temperature determined as discussed above; 1-2 minutes), and extension (72 C for 1 minute). The final extension step is generally carried out for 4 minutes at 72 C and may be followed by an indefinite (0-24 hour) step at 4 C

Several techniques for detecting PCR products quantitatively without electrophoresis may be useful according to the invention. One of these techniques, for which there are commercially available kits such as Taqman™ (Perkin Elmer, Foster City, Calif.), is performed with a transcript-specific antisense probe. This probe is specific for the PCR product (e.g. a nucleic acid fragment derived from a gene of interest) and is prepared with a quencher and fluorescent reporter probe complexed to the 5′ end of the oligonucleotide. Different fluorescent markers can be attached to different reporters, allowing for measurement of two products in one reaction. When Taq DNA polymerase is activated, it cleaves off the fluorescent reporters of the probe bound to the template by virtue of its 5′ -to-3′ nucleolytic activity. In the absence of the quenchers, the reporters now fluoresce. The color change in the reporters is proportional to the amount of each specific product and is measured by a fluorometer; therefore, the amount of each color can be measured and the PCR product can be quantified. The PCR reactions can be performed in 96 well plates so that multiple samples can be processed and measured simultaneously. The Taqman™ system has the additional advantage of not requiring gel electrophoresis and allows for quantification when used with a standard curve.

B. Detection of a Protein Sequence of Interest

1. Preparation of Antibodies

Antibodies specific for the proteins of interest of the invention are useful for protein purification and detection. By antibody, we include constructions using the binding (variable) region of such an antibody, and other antibody modifications. Thus, an antibody useful in the invention may comprise a whole antibody, an antibody fragment, a polyfunctional antibody aggregate, or in general a substance comprising one or more specific binding sites from an antibody. The antibody fragment may be a fragment such as an Fv, Fab or F(ab′)₂ fragment or a derivative thereof, such as a single chain Fv fragment. The antibody or antibody fragment may be non-recombinant, recombinant or humanized. The antibody may be of an immunoglobulin isotype, e.g., IgG, IgM, and so forth. In addition, an aggregate, polymer, derivative and conjugate of an immunoglobulin or a fragment thereof can be used where appropriate.

Although a protein product (or fragment or oligopeptide thereof) of a gene of interest of the invention that is useful for the production of antibodies does not require biological activity, it must be antigenic. Peptides used to induce specific antibodies may have an amino acid sequence consisting of at least five amino acids and preferably at least 10 amino acids. Preferably, they should be identical to a region of the natural protein and may contain the entire amino acid sequence of a small, naturally occurring molecule. Short stretches of amino acids corresponding to the protein of interest of the invention may be fused with amino acids from another protein such as keyhole limpet hemocyanin or GST, and antibody will be produced against the chimeric molecule. Procedures well known in the art can be used for the production of antibodies to the proteins of interest of the invention.

For the production of antibodies, various hosts including goats, rabbits, rats, mice etc . . . may be immunized by injection with the protein products (or any portion, fragment, or oligonucleotide thereof which retains immunogenic properties) of the genes of interest of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

a. Polyclonal antibodies.

The antigen protein may be conjugated to a conventional carrier in order to increase its immunogenicity, and an antiserum to the peptide-carrier conjugate will be raised. Coupling of a peptide to a carrier protein and immunizations may be performed as described (Dymecki et al., 1992, J. Biol. Chem., 267: 4815). The serum can be titered against protein antigen by ELISA (below) or alternatively by dot or spot blotting (Boersma and Van Leeuwen, 1994, J. Neurosci. Methods, 51: 317). At the same time, the antiserum may be used in tissue sections prepared as described. A useful serum will react strongly with the appropriate peptides by ELISA, for example, following the procedures of Green et al., 1982, Cell, 28: 477.

b. Monoclonal antibodies.

Techniques for preparing monoclonal antibodies are well known, and monoclonal antibodies may be prepared using a candidate antigen whose level is to be measured or which is to be either inactivated or affinity-purified, preferably bound to a carrier, as described by Arnheiter et al., 1981, Nature, 294;278.

Monoclonal antibodies are typically obtained from hybridoma tissue cultures or from ascites fluid obtained from animals into which the hybridoma tissue was introduced.

Monoclonal antibody-producing hybridomas (or polyclonal sera) can be screened for antibody binding to the target protein.

2. Antibody Detection Methods

Particularly preferred immunological tests rely on the use of either monoclonal or polyclonal antibodies and include enzyme-linked immunoassays (ELISA), immunoblotting and immunoprecipitation (see Voller, 1978, Diagnostic Horizons, 2:1, Microbiological Associates Quarterly Publication, Walkersville, Md.; Voller et al., 1978, J. Clin. Pathol., 31: 507; U.S. Reissue Pat. No. 31,006; UK Patent 2,019,408; Butler, 1981, Methods Enzymol., 73: 482; Maggio, E. (ed.), 1980, Enzyme Immunoassay, CRC Press, Boca Raton, Fla.) or radioimmunoassays (RIA) (Weintraub, B., Principles of radioimmunoassays, Seventh Training Course on Radioligand Assay Techniques, The Endocrine Society, March 1986, pp. 1-5, 46-49 and 68-78). For analysing plants for the presence or absence of a protein of interest according to the present invention, immunohistochemistry techniques may be used. It will be apparent to one skilled in the art that the antibody molecule may have to be labelled to facilitate easy detection of a target protein. Techniques for labelling antibody molecules are well known to those skilled in the art (see Harlow and Lane, 1989, Antibodies, Cold Spring Harbor Laboratory).

Multiplication of Plant Calluses and Selection of Plant Calluses Comprising at Least One Genetic Transformation Event

Plant calluses are multiplied by growing them on a suitable growth media containing plant hormones that regulate and stimulate cell differentiation. For example, immature embryos 1.0 to 1.2 mm in length (9 to 14 days after pollination) are cultivated on LS-AS medium (Sigma, St. Louis, Mo.) in the dark at 25° C. for 3 days, then transferred onto LSD 1.5 medium (Sigma, St. Louis, Mo.) supplemented with phosphinotricine at 5 mg/l and cefotaxime at 250 mg/l, in the dark at 25° C. for 2 weeks and, finally, placed on LSD 1.5 medium supplemented with phosphinotricine at 10 mg/l and cefotaxime at 250 mg/l, in the dark at 25° C. for 3 weeks. The generated type I calli are isolated, fragmented and transferred onto LSD 1.5 medium supplemented with phosphinotricine at 10 mg/l and cefotaxime at 250 mg/l, in the dark at 25° C. for 3 weeks. Then, the type I calli which proliferate, are isolated and placed on LSZ medium (Sigma, St. Louis, Mo.) supplemented with phosphinotricine at 5 mg/l and cefotaxime at 250 mg/l, under a 16 hours light/8 hours darkness photoperiod at 25° C. for 2 to 3 weeks. The regenerated plantlets are then transferred onto LSF ½ medium (Sigma, St. Louis, Mo.) under a 16 hours light/8 hours darkness photoperiod at 25° C. for 1 to 2 weeks in a growth chamber and further in a phytotron and then a greenhouse.

The preparation and culture of plant calli and selection of calli which express the transgene of interest may be performed using techinques which are well known to those of skill in the art. Such techniques may be found, for example, in Freeling and Walbot, 1994, The Maize Handbook, Springer Verlag, New York, Inc.

Pollination of Whole Transgenic plants By Self- or Free Pollination

In one embodiment, pollination is carried out by hand, wherein the pollen is collected from the flowers or the male part of the plant, and then applied, for example with a small paintbrush, to the female part of the plant. For corn, this involves taking pollen from the apical flowers and applying the pollen to the silks. Both self and free-spollination are carried out according to this method.

In another embodiment, self-pollination, the pollination of one plant by pollen of that plant falling onto the female part of that same plant, is performed, or plants are incubated under conditions that promote self-pollination. Self-pollination often happens naturally in the field.

In another embodiment, free pollination is performed. Free pollination is a form of pollination whereby the pollen from a plant is carried by a vector, for example the wind, insects, or birds, to the female part of a different plant.

Harvesting seeds

Seeds are harvested in a variety of ways, either mechanically or by hand, such methods being well known to the skilled person per se, and including collecting seeds using a combine harvester.

Sowing Seeds

Seeds are sown with a seed drill, as is commonly known to those skilled in the art.

Post-Harvest Phenotypic Sorting

Post-harvest phenotypic sorting is carried out mechanically or manually, and preferably mechanically, for example by sorting seeds by a distinguishing phenotypic characteristic. For example, the transgenic seeds are one color, for example red, and the non-transgenic seeds are another color, for example, yellow. In this case, the seeds pass through an apparatus on a conveying means, and a detector detects the color of the seed, for example by colorimetry, and then operates a separating channel as a function of the color detected.

Castrating or Pollarding a Plant

A plant is castrated or pollared by cutting off the top of the plant that bears the male part of the plant. In corn, for example, this is typically accomplished by hand with a pair of shears, or can be carried out mechanically by a tractor bearing a set of rotating blades that will sever the heads of the plant at the same height.

Genes of Interest and Proteins of Interest According to the Invention

Preferred proteins of interest for use with the present invention include reporter molecules, such as firefly luciferase (GenBank #M15077), glucuronidase (GUS)(Genbank #AAC74698), green fluorescent protein (GFP) (GenBank #E17099), and enhanced versions thereof, particularly for use in optimizing the parameters of this production system. Proteins useful according to the methods of the invention also include but are not limited to proteins that are useful according to the invention, such as receptors, enzymes, ligands, regulatory factors, and structural proteins. Therapeutic proteins including nuclear proteins, cytoplasmic proteins, mitochondrial proteins, secreted proteins, plasmalemma-associated proteins, serum proteins, viral antigens and proteins, bacterial antigens, protozoal antigens and parasitic antigens are also usefil according to the invention.

Therapeutic proteins useful according to the invention also include lipoproteins, glycoproteins, phosphoproteins. Proteins or polypeptides which can be expressed using the methods of the present invention include hormones, growth factors, neurotransmitters, enzymes, clotting factors, apolipoproteins, receptors, drugs, oncogenes, tumor antigens, tumor suppressors, structural proteins, viral antigens, parasitic antigens and bacterial antigens. Specific examples of these compounds include proinsulin (GenBank #E00011), growth hormone, dystrophin (GenBank # NM_007124), androgen receptors, insulin-like growth factor I (GenBank #NM_00875), insulin-like growth factor II (GenBank #X07868) insulin-like growth factor binding proteins, epidermal growth factor TGF-α(GenBank #E02925), TGF-β (GenBank #W008981), PDGF (GenBank #NM_002607), angiogenesis factors (acidic fibroblast growth factor (GenBank #E03043), basic fibroblast growth factor (GenBank #NM_002006) and angiogenin (GenBank #M11567)), matrix proteins (Type IV collagen (GenBank #NM_000495), Type VII collagen (GenBank #NM_000094), laminin (GenBank #J03202), phenylalanine hydroxylase (GenBank #K03020), tyrosine hydroxylase (GenBank #X05290)), oncogenes (ras (GenBank #AF 22080), fos (GenBank #kO0650), myc (GenBank #J00120), erb (GenBank #X03363), src (GenBank #IAH002989), sis GenBank #M84453), jun (GenBank #J041 11)), E6 or E7 transforming sequence, p53 protein (GenBank #AH007667), Rb gene product (GenBank #ml9701), cytokine receptor, III (GenBank #m54933), IL-6 (GenBank #eO4823), IL-8 (GenBank #119591), viral capsid protein, and proteins from viral, bacterial and parasitic organisms which can be used to induce an immunologic response, and other proteins of useful significance in the body.

The compounds which can be incorporated are only limited by the availability of the nucleic acid sequence for the protein or polypeptide to be incorporated. One skilled in the art will readily recognize that as more proteins and polypeptides become identified they can be integrated into the DNA constructs of the invention and used to produce transgenic plants, useful for amplification of a gene of interest and overproduction of a protein of interest, according to the methods of the present invention.

B. Nucleotide Sequences Useful According to the Invention

1. Genes Encoding Toxins

Examples of genes useful in the invention include those encoding such agents including but not limited to genes encoding diphtheria toxin, Pseudomonas exotoxin, cholera toxin, pertussis toxin, etc., as follows. Diphtheria toxin-IL2 fusions for inhibition of HIV-1 infection (Zhang et al., 192, Jour. Acquired Immune Deficiency Syndrome 5:1181); Diphtheria toxin A chain for inhibition of HIV viral production (Harrison et al., 1992, AIDS Res. Hum. Retro. 8:39 and Curel et al., 1993, Hum. Gene Ther. 4:71); Diphtheria toxin A chain-liposome complexes for suppression of bovine leukemia virus infection (Kakidani et al., 1993, Microbiol. Immunol. 37:713); Diphtheria Toxin A chain gene coupled with immunoglobulin enhancers and promoters for B-cell toxicity (Maxwell et al., Cancer Res., 1991, 51:4299); Tat- and Rev- activated expression of a diphtheria toxin A gene (Harrison, 1991, Hum. Gene Ther. 2:53); Diphtheria toxin-CD4 fusion for killing of HIV-infected cells (Auilo et al., 1992, Eur. Mol. Biol. Org. Jour. 11:575).

Other toxins which are useful according to the invention include but are not limited to the following. Conditionally toxic retroviruses are disclosed in Brady et al., 1994, Proc. Nat. Aca. Sci. 91:365 and in Caruso et al., 1992, Bone Marrow Transplant, 9:187. Toxins against EBV infection are disclosed in Harris et al., 1991, Cell. Immunol. 134:85, and against poliovirus in Rodriguez et al., 1992, Jour. Virol. 66:1971. Toxins against influenza virus are disclosed in Bron et al., 1994, Biochemistry 33:9110.

2. Genes Encoding Immunoactive Agents

Another agent useful according to the invention includes immunoactive agents, i.e., agents which combat viral infections or production by activating an immune response to the virus. Such agents include but are not limited to cytokines against viruses in general (Biron, 1994, Curr. Opin. Immunol. 6:530); soluble CD4 against SIV (Watanabe et al., 1991, Proc. Nat. Aca. Sci. 88:126); CD4-immunoglobulin fusions against HIV-1 and SIV (Langner et al., 1993, Arch. Virol. 130:157); CD4(81-92)-based peptide derivatives against HIV infection (Rausch et al., 1992, Biochem. Pharmacol. 43:1785); lympho-cytotoxic antibodies against HIV infection (Szabo et al., 1992, Acta. Virol. 38:392); IL-2 against HIV infection (Bell et al., 1992, Clin Exp. Immunol. 90:6); and anti-T cell receptor antibodies against viruses in general (Newell et al., 1991, Ann. N.Y. Aca. Sci. 636:279).

3. Genes Encoding Anti-Viral Drugs

Genes encoding anti-viral agents useful according to the invention include genes encoding drugs having anti-viral activity and which are the direct product of a gene or are a product of a gene encoding a precursor of the drug, the drug then being synthesized by a biosynthetic pathway in the cell. Targets of drug intervention in the replicative cycle of, for example, a retrovirus, include (1) binding and entry, (2) reverse transcriptase, (3) transcription and translation, and (4) viral maturation and budding. Representative inhibitors of viral binding and entry for HIV include recombinant soluble CD4, immunoadhesions, peptide T, and hypericin. Nucleoside reverse transcriptase inhibitors include zidovudine, didanosine, zalcitabine, and starudine. Foscamet, tetrahydroimidazobenzodiazepinethione compounds, and nevirapine are some non-nucleoside reverse transcriptase inhibitors. Inhibitors of transcription and translation include antagonists of the TAT gene and GLQ223. Castanospermine and protease inhibitors interfere with viral budding and maturation. Such drugs include but are not limited to nucleoside or nucleotide analogs and products of a cellular biosynthetic pathway such as described in Harrell et al., 1994, Drug Metab. Dispos. 22:124 (deoxy-guanine); Fillon et al., 1993, Clin. Invest. Med. 16:339 (dauno-rubicin); Ohrvi et al., 1990, Nucleic Acids Symp. 26:93 (anti-viral nucleosides); Hudson et al., 1993, Photochem. Photobiol. 57:675 (thiarubines); Salhany et al., 1993, Jour. Biol. Chem. 268:7643 (pyridoxal 5′ -phosphate); Damaso et al., 1994, Arch. Viral. 134:303 (cyclosporin A); Gallicchio et al., 1993. Int. Jour. Immunol. 15:263 (dideoxynucleoside drugs); and Fiore et al., 1990, Biol. Soc. Ital. Biol. Sper. 66:601 (AZT).

For many of these and other non-plant proteins, the nucleotide sequence encoding the protein is preferably optimized for expression in plants, e.g., by introducing one or more codons degenerate to the corresponding native codon. Other plant-optimization measures for coding sequences include removal of spurious mRNA processing signals such as polyadenylation signals, splicing sites, and transcription termination signals, removal of mRNA destabilizing sequences, removal of the cytosine methylation motif“CCGG”, modifying the translation start site and introducing a C-terminal KDEL signal, such as SEKDEL (Ser-Glu-Lys-Asp-Glu-Leu), which presumably aids return of the nascent protein to the endoplasmic reticulum for processing. A nucleotide sequence of interest of the present invention can be provided as its wild-type sequence. Alternatively, a synthetic sequence, such as a “plant-optimized” sequence mentioned above can be employed. A nucleotide sequence having a high degree of homology to these sequences, so that the encoded amino acid sequence remains substantially unchanged, are also contemplated. In particular, sequences at least 80%, more preferably 90%, homologous with an aforementioned nucleotide sequence are contemplated. It should be noted, however, that only that those epitopes of an expressed antigenic protein essential for generating the desired immune response need be present in the translated molecule. Accordingly, C- and/or N-terminal fragments, including portions of fusion proteins, presenting the essential epitopes are contemplated within the invention. Such fragments can be encoded in a vector construct of the invention or can be generated in vivo or in vitro by post-translation cleavage processes.

Assesing Biomass

The present invention relates to a method for increasing the transgenic biomass of a transgenic plant, and thus, further relates to methods for assessing transgenic biomass. Transgenic biomass may be assessed from the whole transgenic plant, or from a portion of the plant of interest. For example, if the transgene is operably linked to a leaf-specific promoter (see above for a description of transgene constructs useful in the present invention), then transgenic biomass may be measured in one or more plant leaves obtained from the transgenic plant by determining the presence of, or measuring transgene expression and/or the amount of transgene product (i.e., the polypeptide encoded by the transgene). If the transgene is operably linked to a seed-specific promoter in, for example, corn, then biomass may be determined by measuring the amount of transgene expressed and/or the amount of transgene product produced in the corn seeds.

Transgenic biomass may be measured using techinques which are well known to those of skill in the art. For example, transgene expression may be measured using techinques including, but not limited to Southern blot analysis, northern blot analysis, and PCR-based techniques. These methods are described above and further described in widely available texts and laboratory manuals (see for example, Ausubel et al., 1995 Short Protocols in Molecular Biology 3^{rd} Ed. John Wiley & Sons, Inc.). The biomass of the transgene expression product may be measured using antibodies specifically designed to recognize one or more epitopes on the protein encoded by the transgene. Such antibodies may be monoclonal or polyclonal, and may be generated as described above. Preferred immunological tests to measure the biomass of protein encoded by the transgene rely on the use of such antibodies and include enzyme-linked immunoassays (ELISA), immunoblotting and immunoprecipitation (see Voller, 1978, Diagnostic Horizons, 2:1, Microbiological Associates Quarterly Publication, Walkersville, Md.; Voller et al., 1978, J. Clin. Pathol., 31: 507; U.S. Reissue Pat. No. 31,006; UK Patent 2,019,408; Butler, 1981, Methods Enzymol., 73: 482; Maggio, E. (ed.), 1980, Enzyme Immunoassay, CRC Press, Boca Raton, Fla.) or radioimmunoassays (RIA) (Weintraub, B., Principles of radioimmunoassays, Seventh Training Course on Radioligand Assay Techniques, The Endocrine Society, March 1986, pp. 1-5, 46-49 and 68-78). For analysing plants for the presence or absence of a protein of interest according to the present invention, immunohistochemistry techniques may be used. It will be apparent to one skilled in the art that the antibody molecule may have to be labelled to facilitate easy detection of a target protein. Techniques for labelling antibody molecules are well known to those skilled in the art (see Harlow and Lane, 1989, Antibodies, Cold Spring Harbor Laboratory).

In addition, biomass may be quantitated, for example, by quantitating the amount of protein produced from transgene expression. Any method suitable for quantitating protein from plants may be useful in the present invention. Sucht methods for quantitating protein expression are well known to those of skill in the art and may be found in, for example, Darbre, 1986 “Analytical Methods” In Practical Protein Chemistry: A Handbook, John Wiley and Sons, N.Y.

The following detailed description indicates, by way of non-limiting example, the preferred embodiments of the present invention.

### EXAMPLES

### Example 1

The genetic transformation of a plant requires the integration of a transgene, the selection of the transformed cells, their multiplication and their differentiation into new plantlets.

The technique of genetically transforming maize, whether using a particle gun or using an agrobacterium as described by Y. Ishida et al. (Nature Biotech volume 14, June 1996, 745-749), involves the multiplication of transformed calluses having a regeneration potential. The first part of the invention consists of amplifying the regeneration number obtained from a genetic transformation event.

The cell biologist is capable of detecting, sampling and isolating each set of cells which is derived from a transformation event because transformed cells are capable of developing on a selective medium (whereas the non-transformed cells and tissues are not capable of proliferating on the selective medium). Subsequently, through successive changes in media comprising suitable phytohormonal balances, genetically modified new plantlets will develop. The biologist identifies the transgenic callus based on the observation of the precise site where it forms. This macroscopic screen does not make it possible to separate two genetic transformation events which have taken place in neighbouring cells. This is why, so as not to risk having various transformation events derived apparently from the same primary callus, biologists usually bring to maturity only one to two plantlets per callus identified.

It is possible to verify whether plants do indeed originate from the same transformation event, by carrying out a molecular analysis of their DNA. By performing a Southern blot with one or more probes that bind to the transgene, it is possible, by choosing the restriction enzymes judiciously, to verify that the transgene(s) is(are) indeed inserted at the same place. Experience shows that an experienced cell biologist performs good selection of the calluses, and that the regenerations produced from the same callus are, in the very great majority of cases, derived from the same transformation event. Consequently, by adding one or two additional subculturings during the conventional phases of transgenic callus multiplication, it becomes possible to increase the regeneration number obtained from a transformation event. The extra cell biology work is minimal, and the protocol is lengthened by only approximately three weeks, out of a method which conventionally lasts on average twenty-nine weeks. Controls by Southern blot analysis, as described above, will make it possible to verify that all the plants obtained are indeed derived from the same transformation event. With respect to the transgene, they are copies, although some somatic clonal variations may occur. It is reasonable to envisage producing in this way about twenty copies of each transformation event, whereby the copies comprise the genetic transformation event or events of interest, including the gene coding for the product of interest.

The techniques for genetically transforming plants which are routinely used do not make it possible to totally control the integration of the transgene, the choice of the integration site, or the number of copies of the transgene. Consequently, it is usual to produce at least 10 and preferably at least 20 primary transformants using a molecular construct whose integration into the genome of the plant is desired, this being so as to be able to select the best transformants (for example, to be sure of the presence of only the desired sequences, or alternatively of correct expression). Conventionally, these newly formed plantlets, termed primary transformants, are, after a short development in vitro, acclimatized and brought to maturity in a green-house. In the case of maize, on the one hand given the disturbances engendered by in vitro culturing on their fertility, and on the other hand in order to avoid any transformation event mixing via pollen, the primary transformants are most commonly pollarded (castrated). The descendants of the transgenic plants are obtained by pollinating the transgenic ears by means of non-transgenic pollen. Given, on the one hand, the varieties of maize used to carry out the genetic transformation and, on the other hand, the stress that they undergo in vitro, the total number of T0 seeds obtained is typically between 50 and 150. In the case of monolocus integration of the transgene(s), which is the most common, only 50% of the seeds (termed T1) formed on the primary transformants are transgenic.

These seeds can then be used like any seed. If one of the transgenes confers resistance to a herbicide, it is easy, by means of this herbicide, to select the plantlets derived from the transgenic seeds. Conventionally, these plants are either self-pollinated, or left to free pollinate, in order to obtain the T2 seeds. With this type of variety which is suited to in vitro culturing, the degree of multiplication by generation is approximately 100-fold. The fact of having prepared about twenty copies of each transformation event makes it possible to obtain approximately 1000 transgenic T1 seeds per event (instead of 50 on average with the conventional technique). This makes it possible to have available a pollen mass which is sufficient for pollinating non-transgenic plants with a minimum amount of work. Culturing performed in the open field, or in a greenhouse, is carried out like conventional production of maize hybrids. In this case, the transgenic plants are used as males, and are sown in a line alternating (in a 4/2 or most commonly a 6/2 configuration) with non-transgenic plants used as females. These transgenic plants are ideally sterile male plants, which decreases the work, otherwise they can be castrated male plants. The plants are sown in a line, and not as a mixture, because it is necessary to be able to treat the male plants with a herbicide in order to eliminate those which have not inherited the transgene (50%). Moreover, sowing in a line makes it possible to handle the differing earliness of the male and female plants.

“Earliness” refers to the time of the year at which the crop comes to maturity. It is common to say for example “early” or “winter” wheat, because the wheat variety has been selected to grow during the winter, and will come to maturity earlier than a more traditional variety of the same plant. The same exists for corn, in that it is common to select corn, depending on the geographic site of production, that will ripen earlier than normal corn, in order to avoid frost for example; or as in the case above, to ensure that the rows of female plants will start to produce floral tillers before the male plants so that they can be castrated (hence transforming them into female plants). In this way, the male plants used for pollination, that have a slower development cycle, will flower afterwards and the pollen will then fall onto the silks of the female plants.

There is a double advantage to this approach:

the amount of biomass is very significantly increased with respect to a culture without female plants. Two experiments in the field have allowed us to multiply the biomass 6-fold: five times more biomass was harvested off the female plants than off the male plants.

the quality of the biomass is much better because the plants used as female are hybrids which have a higher added agronomic value in comparison with the male plants. It is possible to use as female plants the maize hybrids which are targeted for use of these transgenes. From the second generation onwards, a biomass is thus obtained which has a quality which is much closer to the future industrial biomass than would have been the case if work had been carried out only with the male plants.

While the proportion of transgenic seeds harvested off the male plants is 75% with the two techniques (transgenic plants alone or hybrid-type culture), only 50% of the seeds harvested off the female plants will be effectively transgenic. In order to find a remedy for this, we suggest combining with the transgene of interest a gene which confers a phenotypic nature which enables post-harvest industrial sorting. It is, for example, possible to modify the coloration of the maize seeds by modifying the enzymes responsible for the biosynthesis of the pigments. We have, ourselves, verified that industrial sorting can be carried out effectively and with very little expense, by mixing maizes of different colorations. After adjustment, it is possible, in one to two passages through industrial sorters, to obtain batches which are more than 95% pure. The sortings, taking into account the machines used, can be carried out without any difficulty and at very low cost, on productions of several tonnes of seeds. The technique is then as follows: after hybrid-type culturing, the male and female plants can be harvested independently, and then sorted via the phenotypic characteristic (i.e., seed color). The production is then entirely transgenic.

The result is given in Table I, in which it is seen that it is possible, using this innovation, to obtain 65 times more biomass, with a quality which is 1.33 times greater with respect to the conventional technique. The expression “quality” corresponds herein to the proportion of transgenic seeds found in the harvested biomass. This is with minor extra costs and an additional delay which is short since it represents approximately 3 weeks out of a total of 52 weeks (49 weeks without the innovation).

In certain embodiments, addition of a gene which confers a specific phenotype to the gene of interest is not desired. According to this embodiment, the biomass obtained is 120 times greater than with the conventional technique; for ⅙ of the biomass, the quality is the same as with the conventional technique, except that there is 20 times the amount, and for ⅚ of the biomass, the quality is inferior by a third.

In Table I, the values given are mean values to be taken as relative values for comparing the two techniques.

Amount: number of maize seeds.

Quality: compared proportion of transgenic seeds. In terms of genetic heritage or background, the hybrids produce seeds which are much closer to industrial systems than the male transgenic plants.

TABLE I

| | | | | Difference |
|---|---|---|---|---|
| | | | Difference | between |
| | | | between | these two |
| | | | the two | techniques |
| | | Technique | techniques | for the two |
| | Conventional | according to | for this | accumulated |
| Generation | technique | the invention | generation | generations |
| T1 | Number of | Number of | Amount: | |
| | maize seeds | maize seeds | 20 X | |
| | produced: | produced: | Quality: | |
| | 100 | 2000 | 1 X | |
| | Proportion of | Proportion of | Delay: | |
| | transgenic | transgenic | plus 3 to | |
| | seeds: 50% | seeds: 50% | 4 weeks | |
| | Total weight | Total weight | | |
| | of seeds: | of seeds: | | |
| | 30 g | 600 g | | |
| | Delay: | Delay: | | |
| | 29 weeks | 32-33 weeks | | |
| Option 1 | Self- | Use of the | Amount: | Amount: |
| No post | pollination | transgenic plants | 6 X | 120 X |
| harvest | of the | for pollinating, | Quality: | Quality: |
| sorting | transgenic | in addition to | 0.66 X | 0.66 X |
| | plants by | themselves, non- | (for the | (for the |
| | themselves | transgenic sterile | additional | additional |
| | | male hybrids used | 5/6, | 5/6, |
| | | as female plants | otherwise | otherwise |
| | | | equivalent | quality |
| | | | quality) | equivalent) |
| | | | Delay: no | Delay: plus |
| | | | difference | 3 to 4 weeks |
| T2 | Biomass | Biomass | | |
| | multiplied | multiplied by 100 | | |
| | by 100 | after elimination | | |
| | after | of the non- | | |
| | elimination | transgenics | | |
| | of the non- | Biomass | | |
| | transgenics | multiplied by 500 | | |
| | Number of | off the female | | |
| | maize seeds | plants | | |
| | produced: | Number of maize | | |
| | 5000 | seeds produced: | | |
| | Proportion of | 100000 male | | |
| | transgenic | plants 500000 | | |
| | seeds: 75% | female plants | | |
| | Total weight | Proportion of | | |
| | of seeds: | transgenic seeds: | | |
| | 1.5 kg | 75% male plants, | | |
| | Delay: | 50% female | | |
| | 20 weeks | plants | | |
| | | Total weight of | | |
| | | seeds: 180 kg | | |
| | | Delay: 20 weeks | | |
| Option 2 | | Idem, but in this | Amount: | Amount: |
| Post- | | case, the non- | 3.25 X | 65 X |
| harvest | | transgenic seeds | Quality: | Quality: |
| sorting with | | are eliminated | 1.33 X | 1.33 X |
| use of | | post-harvest | Delay: | Delay: |
| phenotypic | | Sorted number of | no | plus 3 to |
| markers | | maize seeds: | difference | 4 weeks |
| associated | | 75000 male plants | | |
| with the | | 25000 female | | |
| transgene of | | plants | | |
| interest | | | | |
| T2 | | Proportion of | | |
| | | transgenic seeds: | | |
| | | 100% male and | | |
| | | female plants | | |
| | | Total weight of | | |
| | | seeds: 97.5 kg | | |
| | | Additional delay: | | |
| | | 1 day | | |

### Example 2

This example describes how to increase transgenic plant biomass.

According to this method, a plant cell is transformed with a vector encoding a gene of interest and a gene encoding for antibiotic resistance, for example kanamycin resistance. In certain embodiments, a cell is also transformed with a nucleic acid encoding a gene which encodes for a protein that produces a visible, phenotypic characteristic that distinguishes one plant from the next, such a seed color, grain size, shape or color, and can be used for post-harvest phenotypic sorting, as described herein. Cells are transformed by electroporation according to methods well known in the art.

Plant calluses are prepared from the transformed cells by utilizing techinques which are well known to those of skill in the art. Such techniques may be found, for example, in Freeling and Walbot, 1994, The Maize Handbook, Springer Verlag, New York, Inc. Calluses comprising transformed cells that contain the gene of interest and the antibiotic resistance gene are selected by growth on media comprising the appropriate antibiotic, for example, kanamycin. The presence of the gene of interest in the selected plant calluses is confirmed with Southern blot analysis, with an appropriate probe, as described herein and in Freeling and Walbot (supra).

Plant calluses comprising at least one genetic transformation event and which are capable of regeneration are multiplied, and whole transgenic T0 plants are regenerated as follows. Plant calluses are grown on a suitable growth media containing plant hormones that regulate and stimulate cell differentiation. For example, immature embryos 1.0 to 1.2 mm in length (9 to 14 days after pollination) are cultivated on LS-AS medium in the dark at 25° C. for 3 days, then transferred onto LSD 1.5 medium supplemented with phosphinotricine at 5 mg/l and cefotaxime at 250 mg/l, in the dark at 25° C. for 2 weeks and, finally, placed on LSD 1.5 medium supplemented with phosphinotricine at 10 mg/l and cefotaxime at 250 mg/l, in the dark at 25° C. for 3 weeks. The generated type I calli are isolated, fragmented and transferred onto LSD 1.5 medium supplemented with phosphinotricine at 10 mg/l and cefotaxime at 250 mg/l, in the dark at 25° C. for 3 weeks. Then, the type I calli which proliferate, are isolated and placed on LSZ medium supplemented with phosphinotricine at 5 mg/l and cefotaxime at 250 mg/l, under a 16 hours light/8 hours darkness photoperiod at 25° C. for 2 to 3 weeks. The regenerated plantlets are then transferred onto LSF ½ medium under a 16 hours light/8 hours darkness photoperiod at 25° C. for 1 to 2 weeks in a growth chamber and further in a phytotron and then a greenhouse. At all stages of growth, the growth medium is supplemented with kanamycin or the appropriate antibiotic or herbicide for the selection process. The medium may also be supplemented with additional or alternative selection components including, but not limited to agromycin and augmentin.

The resulting T0 plants are pollinated with non-transgenic pollen by applying the non-transgenic pollen with a paintbrush to the female parts of the T0 plants.

The resulting T1 seeds that have integrated at least one transgene of interest are harvested by collecting seeds using a combine harvester or by hand harvesting methods.

The transgenic T1 seeds are sown with a seed drill. The plants which result from the sowing of the transgenic T1 seeds are pollinated, either by self-pollination or by free pollination to produce T2 seeds. The resulting T2 transgenic seeds are harvested by collecting seeds with a combine harvester or by hand.

The transgenic biomass of the T2 seeds may subsequently be determined using techniques which are well known to those of skill in the art as described above.

1. A method for increasing transgenic plant biomass, comprising the steps of:
(a) providing plant calluses which contain at least one genetic transformation event and which are capable of regeneration,
(b) multiplying said plant calluses;
(c) regenerating whole transgenic T0 plants from said plant calluses;
(d) pollinating said T0 plants with non-transgenic pollen;
(e) harvesting T1 seeds that have integrated at least one transgene of interest;
(f) sowing said transgenic T1 seeds and pollinating the plants which result therefrom, either by self-pollination or by free pollination to produce T2 seeds; and
(g) harvesting the T2 transgenic seeds.

2. The method of claim 1 wherein prior to said regeneration step, calluses comprising at least one genetic transformation event of interest are selected.

3. The method of claim 1, further comprising the step of post-harvest phenotypic sorting of said T2 seeds.

4. The method of claim 1, wherein said method is performed using plants used only as male plants and plants used only as female plants.

5. The method of claim 3, wherein said sorting is carried out on T2 seeds originating from a plant used only as a female.

6. The method of claim 1, wherein said transgenic T2 seeds have a coloured phenotype which is different from non-transgenic T2 seeds.

7. The method of claim 4, wherein said T2 seeds originating from the plants used as male plants and said T2 seeds originating from the plants used only as female plants are harvested independently from each other.

8. The method of claim 1, wherein said plant is allogamous.

9. The method of claim 1, wherein said plant is maize.

10. The method of claim 1, wherein said primary transformants are pollarded or castrated before pollination with non-transgenic pollen.

11. The method of claim 1, wherein said transgenic T1 seeds are sown, cultivated and used as male plants.

12. The method of claim 11, wherein said transgenic T1 seeds are sown in a line alternating in a 4/2 or in a 6/2 configuration, with non-transgenic plants as female plants.

13. The method of claim 12, wherein said female non-transgenic plants are sterile male plants.

14. The method of claim 12, wherein said female non-transgenic plants are castrated.

15. The method of claim 12, wherein said female plants have a high agronomic value compared with the male plants.

16. The method of claim 1, wherein said multiplication step results in the production of at least ten transgenic T0 plants, wherein each of said transgenic T0 plants comprises one of said genetic transformation events.

17. The method of claim 1, wherein said multiplication step results in the production of at least twenty transgenic T0 plants wherein each of said transgenic T0 plants comprises one of said genetic transformation events.
